(19) United States
(12) Patent Application Publication
(10) Pub. No.: US 2014/0121473 A1
(43) Pub. Date: May 1, 2014

(54) VITAL SIGN MONITORING SYSTEM FEATURING ELECTRONIC DIAPER

(71) Applicants: Matt BANET, Kihei, HI (US); Marshal DHILLON, San Diego, CA (US); Robert HUNT, Vista, CA (US)

(72) Inventors: Matt BANET, Kihei, HI (US); Marshal DHILLON, San Diego, CA (US); Robert HUNT, Vista, CA (US)

(21) Appl. No.: 14/066,539

(22) Filed: Oct. 29, 2013

Related U.S. Application Data
(60) Provisional application No. 61/720,786, filed on Oct. 31, 2012.

(51) Int. Cl. A61B 5/00; A61B 5/08; A61B 5/01; A61B 5/0295

(52) U.S. Cl. CPC A61B 5/6808; A61B 5/0295; A61B 5/0809; A61B 5/01; A61B 5/0015
USPC 600/301

(57) ABSTRACT

The invention provides a system for monitoring an infant or adult patient that includes a garment configured to attach to the infant, and a control module connected to the garment featuring: i) a first sensor that measures HR or a parameter used to determine HR; ii) a second sensor that measures RR or a parameter used to determine RR; iii) a third sensor configured to monitor a PP parameter; and iv) a wireless transmitter configured to receive and wirelessly transmit information from the first, second, and third sensors.

### CROSS REFERENCES TO RELATED APPLICATIONS

This application claims the benefit of U.S. Provisional Application No. 61/720,786, filed Oct. 31, 2012.

### STATEMENT REGARDING FEDERALLY SPONSORED RESEARCH OR DEVELOPMENT

Not Applicable

### BACKGROUND OF THE INVENTION

1. Field of the Invention

This invention relates to a system for monitoring vital signs featuring an electronic diaper.

2. Description of the Related Art

Conventional infant monitoring systems typically include a crib-side camera and microphone for capturing images and sounds generated by the infant, and a 1-way wireless system for transmitting these images to a remote display that can be viewed by a family member (e.g. a parent). With such a system, the parent can be removed from the crib and still determine whether the infant is sleeping, crying, or moving about. Typically such systems include viewing devices that are custom-made, hand-held, and feature a simple display for rendering images of the infant and a speaker system for projecting their sounds.

Vital signs, such as heart rate (HR) respiration rate (RR), are sometimes measured from an infant in a hospital or medical clinic. A vital signs monitor, typically featuring a form factor similar to that of a desktop computer, measures an electrocardiogram (ECG) from the infant to determine HR. Such a measurement requires attaching disposable adhesive electrodes to the infant's torso, and then connecting these to an ECG system within the vital signs monitor using a collection of electrical leads. The monitor can also measure RR with a technology called impedance pneumography (IP) which relies on the same electrodes used for ECG-based measurements of HR. In IP measurements one electrode typically injects a low-amperage (e.g. 1 mA) current modulated at a high frequency (e.g. 50 kHz). Breathing-induced impedance changes in the infant's thorax create a measurable voltage change when combined with the injected current. The voltage signal can then be analyzed with signal-processing algorithms to determine RR. Typically HR, RR, and other vital signs are measured from an infant in a neo-natal intensive care unit (NICU).

Vital signs can also be monitored from the infant outside of the NICU, e.g. during a medical check-up. However during such visits infants tend to move and squirm about, making it difficult to measure vital signs such as HR and RR.

Most infants wear diapers that collect urine and fecal matter, with a typical infant using as many as 5-10 diapers every day. Reusable diapers are typically composed of cloth materials, whereas disposable diapers are typically composed of a combination of plastic and cotton-like materials that collect and absorb the infant's waste. Disposable diapers come in many forms, but in general the American market is dominated by the Huggies and Pampers brands, which are developed and marketed by, respectively, Kimberly-Clark and Proctor and Gamble. In total, about 2 billion disposable diapers are deposited in America's landfills each year.

### SUMMARY OF THE INVENTION

The present invention provides an Internet-based monitoring system featuring an ‘electronic diaper’ that collects the following information from a patient: images; sounds; numerical data and physiological waveforms describing HR and RR; motion-related events, including posture; and whether or not the infant has urinated or defecated in the diaper. For this invention the ‘patient’ wears the electronic diaper, and can be either an infant or adult. The system features three primary components. First, an electronic diaper featuring a reusable shell and disposable insert attaches to the patient like a conventional diaper. The reusable shell includes at least two conductive electrodes which are embedded in an inner lining and contact the patient, typically on each side about the pelvis bone. The electrodes are typically made from a conductive rubber or fabric, and connect to a battery-powered control module located in a front portion of the reusable shell. The control module is operated by a programmable microcontroller and features a small-scale, low-power ECG circuit that processes electrical signals collected by the electrodes to determine an ECG waveform. A three-axis accelerometer within the control module simultaneously measures signals related to the patient's motion (e.g. crawling), posture (e.g. standing, lying down), and breathing-induced movement of the infant's belly. Sensors that monitor temperature and moisture are embedded in a lower portion of the reusable shell, just underneath the disposable insert, and detect signals related to urine and feces. These signals are typically time-dependent thermal signals which can be processed as described in detail below to determine parameters that are referred to herein as ‘PP status’ parameters. Such parameters, as used herein, describe a ‘PP event’, which is when the patient urinates or defecates in their diaper. The microcontroller within the control module collects digital representations of these signals, and then ports them through a wireless peer-to-peer interface for further analysis, as described below.

A second component of the invention is a monitoring module typically connected to an infant's crib. For adult patients, the monitoring module can attach, e.g., to a nightstand or bed. The monitoring module typically features a single-board computer and wireless system that collect data transmitted by the control module within the electronic diaper. Both waveform and numerical data are typically sent in a packetized form that is decoded using software operating on the single-board computer. A webserver software program is also coded within this platform and analyzes and then avails information received by the monitoring module to other computing platforms (e.g. computer, cellular phone) connected to the Internet. Such computing platforms typically receive information served by the webserver through a wireless interface. The single-board computer also operates algorithms that process signals measured by the various sensors within the electronic diaper to determine parameters such as HR, RR, posture, and PP status. The monitoring module also includes an embedded camera (e.g. a conventional web camera) and microphone that collect images and sounds from the infant, and then uses the webserver to avail this information to external Internet-connected devices.

The third component of the invention is a ‘downloadable’ software application that operates on a variety of Internet-connected computing platforms to receive and display information from the webserver. Parents of the infant, for example, can download the software application from a website, e.g. one associated with a company providing the above-mentioned components, or a website (e.g. Apple's iTunes Store) that provides multiple software applications that operate on specific devices (e.g. the iPhone or iPad). The software application typically features a graphical user interface (GUI) that renders information collected by the webserver, e.g. images and sounds from the patient, vital signs, PP parameters, motion-related information, and plots of time-dependent waveforms (e.g. ECG waveforms) indicating the patient's real-time physiological status. In addition, the software application may include an ‘alarm module’ that processes one or more of the above-mentioned parameters to generate an audio/visual alarm in the event that the patient is in distress. For example, the alarm module can generate an alarm if the patient's HR or RR values exceeded a pre-determined threshold, or if the PP parameter indicates that the diaper was soiled. The alarm module can also process a collection of parameters, or trends in these parameters, to determine and possibly predict a relatively complex and dangerous physiological state. In particular, the alarm module includes an algorithm for monitoring trends in HR and RR to predict the onset of sudden infant death syndrome (SIDS), which occurs in about 1 out of every 2000 infants.

The software application can render both real-time and historical information. And because it is accessible through the Internet, the application may be viewed by either the patient's parents, or someone associated with them in another capacity, e.g. another family member or pediatrician. Here, the system may include a website that features separate interfaces (e.g. a ‘family’ interface and a ‘clinician’ interface) that are accessed using a specific username/password. Such a system allows remote family members to view the patient, and also facilitates a ‘virtual check-up’ wherein a clinician can monitor the patient's cardio-pulmonary behavior by viewing time-dependent waveforms and trends in parameters like HR and RR. Additionally, because the electronic diaper includes motion sensors, vital signs and their associated waveforms can be monitored when the patient is relatively motion-free, thus increasing the likelihood that the measured physiological data is not corrupted by motion.

In typical applications, the system according to the invention is used much like conventional infant monitoring systems, only with the distinct advantage that it additionally measures real-time physiological information. For example, the system can be installed so that parents can view images, sounds, vital signs, and PP parameters from the infant using their existing cellular telephone, tablet computer, or laptop computer. These devices can be located at the parent's bedside so that the infant can be monitored during normal sleeping hours. In the unlikely event that a life-threatening physiological event occurs, the software application's alarm module can sound an alarm, allowing the parents or medical clinician to take appropriate action. In another application, the electronic diaper and monitoring module could accompany the infant to a day-care facility, allowing the parent to view their child while at work. In yet another application, a remote family member or local nurse can monitor an aging relative located in an assisted-living facility. In general, using the system described herein, both infant and adult patients can be monitored with a variety of off-the-shelf computing devices from virtually any location having access to the Internet.

More specifically, in one aspect, the invention provides a system for monitoring a patient that includes a garment configured to attach to the patient, and a control module connected to the garment featuring: i) a first sensor that measures HR, blood pressure, and blood oxygen content (SpO2) or a parameters used to determine these properties; ii) a second sensor that measures RR or a parameter used to determine RR; iii) a third sensor configured to monitor a PP parameter; and iv) a wireless transmitter configured to receive and wirelessly transmit information from the first, second, and third sensors. The control module interfaces to a monitoring module, configured to receive information from the first, second, and third sensors through the wireless transmitter, which includes: i) a processing component that processes information generated by the first, second, and third sensors; and ii) a computing component configured to avail content determined by the processing component on a network. A software application operating on a remote computer connects to the network and receives and then displays content availed by the computing component, or parameters calculated therefrom.

In preferred embodiments, the garment is a diaper featuring an outer component configured to attach to the lower portion of the patient's torso, and an inner component which includes an absorbent material configured to contact the patient's skin. Typically the diaper includes at least two conductive electrodes, each made from a conductive material, attached to the outer component and configured to contact the patient's skin. The first sensor can feature an ECG sensor that connects to the conductive electrodes to receive electrical signals, and then processes these signals with a collection of differential amplifiers and analog filters to generate and ECG waveform. In an alternate embodiment, the first sensor features an optical sensor that typically includes a photodiode and a light source (e.g. a light-emitting diode, or LED). Here, the optical sensor can measure a photoplethysmogram (PPG) from the patient, which is a time-dependent waveform indicating blood flow in an artery or capillary located close to the surface of the infant's skin. Algorithms process either (or both) of the ECG and PPG waveforms using techniques described in detail below to determine HR. Additionally, a low-frequency envelope indicating RR is often mapped onto one or both of the ECG and PPG waveforms. This envelope can thus be monitored with standard signal processing techniques to determine RR, as is described in more detail below. PPG waveforms measured with both red and infrared LEDs can also be analyzed to determine the infant's value of SpO2 using known techniques in the art.

In another embodiment, the second sensor within the control module is an accelerometer (typically a three-axis accelerometer) that measures a time-dependent waveform indicating the patient's motion. For example, the accelerometer can measure a time-dependent waveform indicating respiratory-induced motion from the torso. Here, the waveform indicates motion measured along an axis of the accelerometer that is approximately normal to the patient's belly (e.g. within +/−30° of a normal vector extending outward from the infant's belly). In another embodiment, the second sensor associated with the control module includes at least one electrode that measures an electrical impedance change from the patient that varies with respiration rate. Such an electrode, for example, is included in an impedance pneumography sensor. This sensor can be included in the same circuit used to measure ECG waveforms.

In another embodiment, the third sensor with the control module features a thermal sensor that measures, e.g., a digital temperature signal indicative of urine and/or feces from the patient (e.g. the PP parameter referred to above). The third sensor can also include a moisture sensor that measures a related PP parameter. Algorithms described in more detail below process signals from these sensors to determine if the patient has, in fact, soiled their diaper.

In preferred embodiments, the wireless transmitters that connect the control module to the monitoring module operate on a protocol based on 802.11 (e.g. WiFi) or 802.15.4 (e.g. Bluetooth or Zigbee). For example, the wireless transmitter can be a Bluetooth low-energy transmitter, which is optimized to improve battery lifetime.

Typically the processing component within the monitoring module is a computer (e.g. a single-board computer) that operates a collection of algorithms and software programs. For example, to determine HR, the computer can operate a beat-picking algorithm that analyzes ECG waveforms from the first sensor. Such an algorithm can be the Pan-Tompkins algorithm, or a derivative thereof, which is described in the following document, the contents of which are fully incorporated herein by reference: A Real-Time QRS Detection Algorithm, Pan et al., IEEE Transactions of Biomedical Engineering, Vol. BME-32, No. 3, March, 1985. In a related embodiment, another algorithm operating in the monitoring module is a breath-picking algorithm that analyzes waveforms modulated by the patient's breathing patterns to determine RR. For example, the breath-picking algorithm can operate a slope-summing function, or a derivative thereof, such as that described in the following document, the contents of which are fully incorporated herein by reference: An Open-Source Algorithm to Detect Onset of Arterial Blood Pressure Pulses, Zong et al., Computers in Cardiology, Vol. 30, 2003. In this document the slope-summing algorithm is applied to a continuous blood pressure waveform to determine heartbeat-induced pulses, but the same methodology can also be applied to waveforms modulated by breathing patterns to measure RR.

In another embodiment, the algorithm is configured to process information from a thermal sensor to determine signals related to a PP parameter. For example, the algorithm can be a curve-fitting algorithm, such as one that fits a time-dependent waveform from the thermal or moisture sensor with an exponential function, or something similar. In a related embodiment, the algorithm involves measuring a mathematical derivative of the time-dependent waveform generated by the thermal or moisture sensor to determine a change in these signals that may be indicative of a PP parameter.

Preferably the monitoring module includes a camera, e.g. a web camera that captures real-time video images of the patient, and a microphone that captures voice signals indicating, e.g., that an infant is crying. Typically the web camera integrates directly with the single-board computer within the monitoring module. In this case, the computer also operates a webserver that serves up content which can be viewed with a remote, Internet-connected device. For example, the content can be one of the following: an image, a vital sign, a time-dependent physiological waveform, a motion waveform, a motion-related parameter, a posture, an indication if the patient is sleeping, or a PP parameter. In embodiments, the webserver connects to a website, from which content van be viewed through an in-home wireless network connected to the Internet. Typically the content can be viewed by any Internet-connected computing platform using the downloadable software application. Such computing platforms include a desktop computer, laptop computer, tablet computer, cellular telephone, smartphone, or similar device. Such systems typically feature a high-resolution video camera that yields high-quality color images of the infant that can be viewed from either home or work. During night, when the infant is typically sleeping, the computing platform can be located by a parent's bedside like a conventional infant monitoring system.

The software application is typically configured to be downloaded from a website operating on the Internet. It preferably includes a GUI that displays an image and at least one of a vital sign, a time-dependent physiological waveform, a motion waveform, a motion-related parameter, a posture, an indication if the infant is sleeping, and a PP parameter.

In preferred embodiment, the software application includes a section to set and/or select alarm parameters, e.g. those associated with vital sign values, PP parameters, whether or not the patient is sleeping, the patient's posture and motion, and time-dependent trends and/or combinations of these properties.

The system can integrate with any Internet-based system, e.g. a website. Preferably the website includes a first user interface associated with the patient's family, and a second user interface associated with a medical clinician. The clinician, for example, can be a pediatrician, a general physician, or a nurse or assistant working at an assisted-living facility for adults. Typically the second interface is also associated with a plurality of patients, allowing the clinician to check up on one patient from a group of patients. This allows, for example, the pediatrician to check the infant's vital signs, waveforms, crawling and/or sleeping behavior, and a variety of other parameters related to the infant's physiology and behavior. For example, during such a procedure the pediatrician could evaluate trends in the infant's HR and RR values and observe their ECG waveforms to detect cardiac abnormalities. Similarly, algorithms operating with the software application can analyze motion waveforms generated by the accelerometer within the electronic diaper to indicate to the pediatrician if the infant is crawling, sleeping, or moving about in a normal manner.

The invention has a number of advantages. In general, it provides real-time monitoring of a patient using a combination of video images, sound, vital signs, motion, and PP parameters. Such information can be processed with sophisticated software normally associated with hospital-grade vital sign monitors to detect and possibly predict when the patient is in need of medical attention, or simply when a diaper needs to be changed. In one sense, the invention brings aspects of sophisticated medical care normally conducted in the NICU to the home environment. This can potentially empower family members to provide more sophisticated care for their own infant, while also providing data that a clinician can use to make an effect, remote diagnosis.

There are also advantages associated with the form factor of the electronic diaper. As described herein, it includes a relative large reusable shell, and a relatively small disposable insert that gets soiled during a PP event. This means only a small part of the diaper gets thrown away after such an event occurs. Ultimately this helps to reduce the substantial waste associated with disposable diapers. Additionally, the disposable insert can be composed exclusively of biodegradable materials which quickly degrade in landfills. This helps reduce the environmental impact of the disposable insert compared to conventional disposable diapers, which typically include plastic materials which can literally take hundreds of years to degrade.

These and other advantages of the invention may be apparent from the following detailed description, and from the claims.

### BRIEF DESCRIPTION OF THE DRAWINGS

FIG. 1 is a high-level schematic drawing of the invention showing an infant wearing the electronic diaper, the monitoring module, and the downloadable software application running on a remote computer;

FIG. 2A is a three-dimensional, exploded view of the disposable insert used in the electronic diaper of FIG. 1;

FIG. 2B is a three-dimensional drawing of the electronic diaper of FIG. 1 featuring a reusable shell and a disposable insert;

FIG. 3A is a schematic drawing of a front side of the circuit board used in the control module of the electronic diaper of FIG. 1;

FIG. 3B is a schematic drawing of a back side of the circuit board used in the control module of the electronic diaper of FIG. 1;

FIG. 4 is a detailed schematic drawing showing hardware and software components used in the control module, monitoring module, and software application;

FIG. 5 is a three-dimensional drawing of the monitoring module attached to a conventional crib and used to monitor an infant;

FIG. 6 is a photograph of a single-board computer used within the monitoring module of FIG. 5;

FIG. 7 is a drawing of a main screen of the downloadable software application operating on a remote computer;

FIG. 8 includes graphs of time-dependent waveforms generated by a temperature sensor in the reusable shell of FIG. 2B and indicating, respectively, a ‘1’ and ‘2’ PP parameter; and

FIG. 9 is a schematic drawing of a web-based system that integrates to the patient monitoring system of the invention.

### DETAILED DESCRIPTION OF THE INVENTION

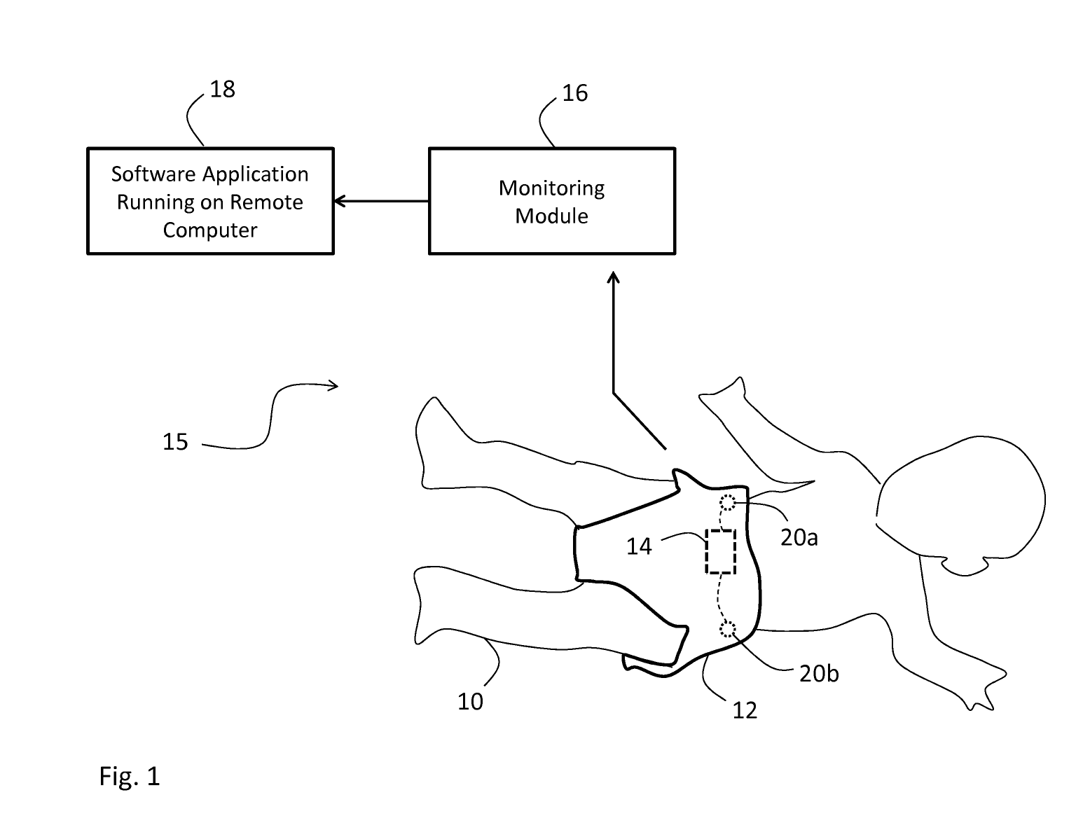

Referring to FIG. 1, an infant monitoring system 15 according to the invention features: i) an electronic diaper 12 worn by an infant 10; ii) a monitoring module 16 that attaches to a conventional crib and receives information from the electronic diaper 12 through a short-range wireless interface; and iii) a downloadable software application 18 operating on a remote computing device that communicates with the monitoring module 16 through a local-area network or, alternatively, the Internet. The infant monitoring system 15 simultaneously collects real-time images, sounds, vital signs, and motion information from the infant 10, and avails this to the downloadable software application 18 through a webserver operating on the monitoring module 16. Ultimately this information is viewed using the downloadable software application 18, which operates on a remote computer, e.g. a conventional tablet computer, laptop computer, cellular phone, or even television with a computer interface. For example, the infant monitoring system 15 can be used to monitor like a conventional ‘baby monitor’, only it has the advantage of collecting and analyzing vital sign information to determine if the infant is approaching a dangerous physiological condition.

The infant monitoring system 15 shown in FIG. 1 and described herein can also be used to monitor any patient wearing the electronic diaper. Such patients, for example, include adult patients, such as those in assisted-living facilities.

A collection of sensors within the electronic diaper 12 measure ECG and motion waveforms from the infant, which are then wirelessly transmitted to the monitoring module 16 for analysis. In this way the electronic diaper 12 serves as a ‘hub’ that collects information from the infant, leaving the bulk of the analysis for a relatively high-power computer operating on the monitoring module 16. This also reduces power consumed by the microcontroller within the control module, thereby improving battery lifetime. To minimize the time required for wireless transmission, and thus further minimize power consumption, the control module typically transmits a version of the waveform that has relatively few data points (i.e. a decimated waveform) to the monitoring module for further processing.

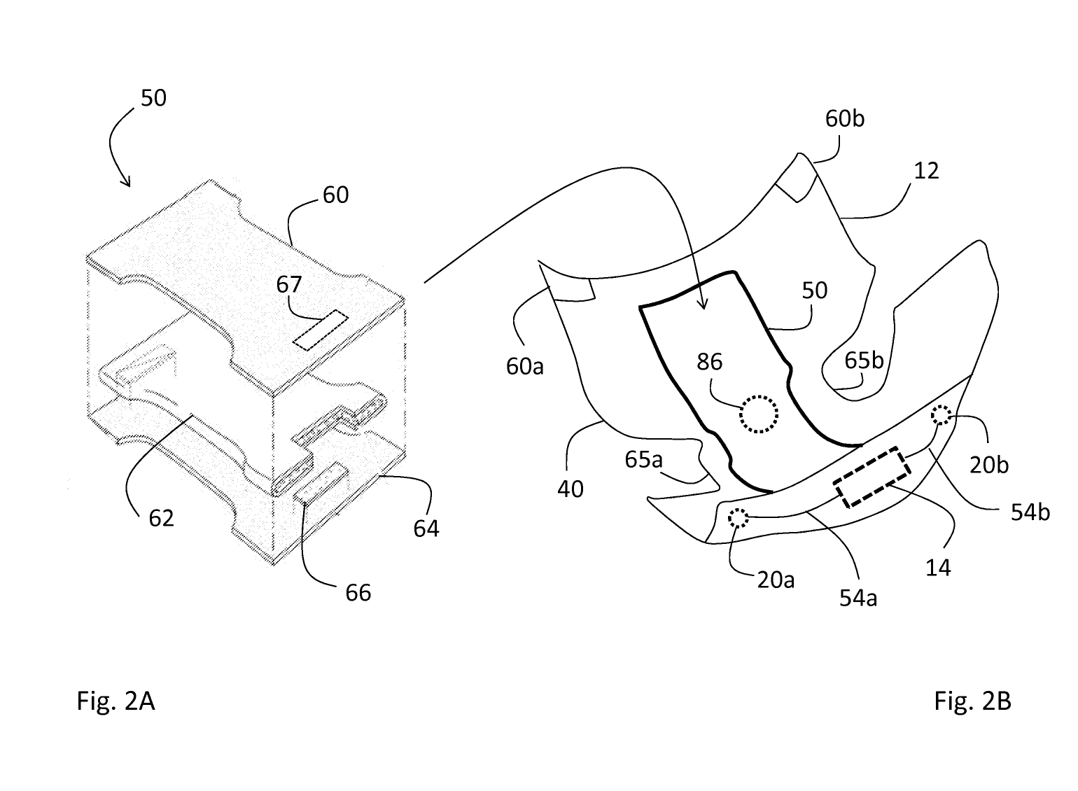

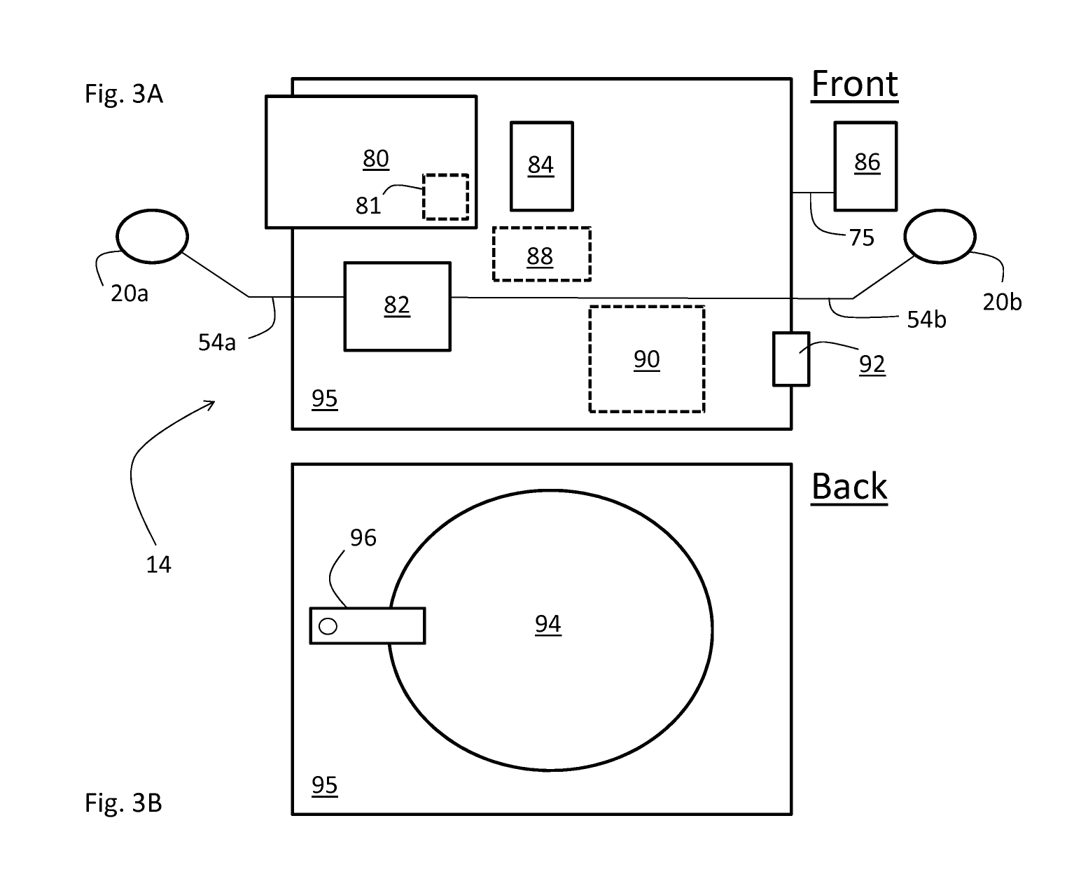

More specifically, in a preferred embodiment as shown in FIGS. 2A, 2B, 3A, 3B, the electronic diaper 12 features a reusable shell 40 and a disposable insert 50. The reusable shell 50 houses a control module 14 that is typically encapsulated in a waterproof plastic container, and connects through a pair of conductive cables 54a, 54b to a pair of electrodes 20a, 20b that are embedded in the material used to construct the reusable shell. For example, the conductive cables 54a, 54b can be conventional insulated wires or flexible circuits, while the electrodes can be patches of conductive rubber or fabric. Such materials typically have an internal resistance of about 100 ohms/cm. Electrodes 20a, 20b are fabricated within the disposable shell 40 so that they contact the infant's skin when the electronic diaper is worn. The control module 14 is the ‘brain’ of the electronic diaper, and features an embedded microcontroller 81; digital ECG circuit 82, accelerometer 84, and temperature 86 sensors; and Bluetooth transmitter 80 that wirelessly transmits information from the electronic diaper over a range of about 10 meters. The microcontroller 81 can be a stand-alone component, or more preferably is embedded within the Bluetooth transmitter 80, which also requires a processor for its operation. Typically it connects to the ECG circuit 82, accelerometer 84, and temperature 86 sensors through conventional computer interfaces, e.g. UART, SPI, or I2C. Embedded analog circuitry (not shown in the figure) filters signals from the digital ECG 82, accelerometer 84, and temperature 86 sensors to remove extraneous noise that may affect accuracy of the various measurements made by the diaper. Additionally, the control module may include additional analog circuitry 88 for other sensors, such as an optical sensor, described in more detail below. A rechargeable Li:ion battery 94 powers each of these components, which are typically surface-mounted on a thin circuit board 95. Typically the battery 94 has a conventional ‘coin cell’ configuration, and is held on the opposite side of the circuit board 95 with a soldered tab 96. The control module 14 also includes power management circuitry 90 that connects to a lead on the battery and regulates voltages required by the above-mentioned electronic components (typically 3.3 and 1.2V). A USB interface 92 allows the control module 14 to plug into a wall outlet (using, e.g., an AC/DC adaptor) or computer (e.g. the monitoring module), and receive power that recharges the Li:ion battery 94. Typically electronic circuitry for recharging the battery is included within the power management circuitry 90.

To minimize the size of the circuit board 95, the digital ECG system 82 is implemented using a single-chip analog front end, such as the ADS 1298 manufactured by Texas Instruments. This integrated circuit combines low-noise amplifiers and high-resolution analog-to-digital converters for multiple channels into a small, low-power electronic package. The ECG circuit 82 connects to separate conductive electrodes 20a, 20b integrated within the reusable shell 40 that, during use, contact opposing sides of the infant. The conductive electrodes, for example, are composed of materials such as conductive rubber or fabric. To measure an ECG waveform, the electrodes 20a, 20b measure weak electrical signals that pass through wires 54a, 54b embedded within the reusable shell 40 to the ECG circuit 82. There, the circuitry described above collectively generates a digital, time-dependent ECG waveform, which is sent to the monitoring module 16 for further processing.

The accelerometer 84 measures motions associated with the infant along three unique, orthogonal axes. Motions along the axis normal to the infant's belly will be heavily influenced by respiratory effort, as most infants are classified as ‘belly breathers’, meaning their stomachs move up and down during the breathing process. The accelerometer 84 detects this process to generate a time-dependent motion waveform which is digitized by an internal analog-to-digital converter to yield a motion waveform. All three time-dependent motion waveforms are affected by different motion-related events. For example, the infant's position relative to gravity will change if the infant is standing up or lying down; this affects the motion waveforms, allowing posture to be determined with a simple algorithm. Motions associated with crawling or rocking will impart a unique, often periodic signal on the motion waveforms, and thus these activities can also be determined by analyzing the motion waveforms. Analysis of the motion waveforms, like that associated with the ECG waveforms, takes place on the monitoring module 16 as described in more detail below.

The electronic diaper additionally includes a thermal sensor 86 located underneath the disposable insert that connects to the control module 14 through a thin wire. The thermal sensor 86 measures properties indicative of a PP event. For example, when such an event occurs, the temperature near the disposable insert typically rises by about 10-20° F. above ambient temperature. The rise of this signal will take place within a few seconds, whiles its decay will depend on whether the infant has urinated in the diaper (causing the temperature signal to decay away relatively fast, as urine is absorbed by the disposable insert) or defecated in the diaper (resulting in a relatively slow decay of the thermal signal). The thermal sensor 86 includes an internal analog-to-digital converter and is calibrated to generate accurate numerical values for temperature levels. The same thermal sensor, or alternatively a separate thermal sensor located near one of the electrodes, can also be used to estimate the infant's skin temperature. Additionally, the control module 14 can connect to a moisture sensor that is typically disposed proximal to the temperature sensor to detect increases moisture levels associated with a PP event. These signals are typically processed collectively with thermal signals, as described above, to determine such an event.

Algorithms operating on the monitoring module 16 can process the ECG waveforms to determine HR and cardiac abnormalities, and process the motion waveforms to determine RR, posture, and information related to how the infant is moving in the crib. Once determined, the webserver operating within the monitoring module 16 avails this information along with that described above to downloadable software application 18 operating on the remote computer. In the rare and unfortunate event that the infant stops breathing, or experiences another physiological abnormality (e.g. a high HR), alarming software operating on the monitoring module will trigger an alarm that gets sent to the remote viewing device. This typically activates auditory and visual alarms, thus alerting the parents and possibly a medical clinician to take the appropriate action.

FIGS. 2A and 2B show the reusable shell 40 and disposable insert 50 of the electronic diaper 12, and how this garment attaches to an infant. The disposable insert 50, shown in FIG. 2A, features a soft, tissue-based hydrophilic layer 60 that contacts the infant's skin and provides comfort while allowing liquids to easily pass into an underlying absorbent core 62, but not return to the infant's skin. With this material the infant's skin stays dry even during a PP event. Typically the hydrophilic layer 60 is about 1-2 mm thick, and consists of a non-woven material that is treated with a surfactant chemical that optimizes its ability to pass liquids. It can also be infused with substances such as topical lotions, Aloe Vera, Vitamin E, Petrolatum, etc., to protect the infant's skin. The absorbent core 62, which lies just underneath the hydrophilic layer 60, is typically constructed of a cellulose-based material and gives the diaper its primary absorbing capacity. Typically the absorption capacity of pulp in the cellulose is around 10 cc of water/gram of pulp when the diaper is in saturated, but less than 2 cc when subjected to 5 KPa of pressure. The thickness of the absorbent code is about 5 mm. Underneath it is an acquisition and distribution layer 64, which is a sub-layer that integrates with the absorbent core 62 and further absorbs liquids to prevent potential leakage. This material typically features sodium polyacrylate, which is a super-absorbent polymer that further improves the capacity and retention of the insert. A pair of Velcro tabs 66, 67 holds the hydrophilic layer 60, absorbent core 62, and acquisition and distribution layer 64 together, while a series of Velcro patches (not shown in the figure) secure the disposable insert to the reusable shell 40. The shell 40 may also include alignment markings that allow the insert 50 to be properly attached.

FIG. 2B shows this component in more detail. It is typically composed of a polyethylene or cloth-like material engineered to stop liquids from leaking out of the diaper. This material should be breathable to keep the infant comfortable, and typically includes a gathered elastic material 65a, 65b where the infant's legs are inserted to provide a seal and further prevent leakage. Velcro tabs 60a, 60b secure the reusable shell to the infant.

The control module 14 and its various sensors are attached directly to the reusable shell 40. Specifically, the module 14 is embedded in the polyethylene or cloth-like material so that it is not visible, although this component may include a small LED that is exposed and periodically blinks indicating the control module 14 is turned on an operational. As described above, the control module 14 attaches to a pair of conductive electrodes 20a, 20b through wires 54a, 54b that allows electrical signals to be collected from the infant and analyzed with the ECG circuit to determine an ECG waveform and, ultimately, HR. The temperature sensor 86 is attached to the surface of the reusable shell near the infant's bottom so that it can adequately detect temperature changes indicating a PP event, and connects to the control module 14 through an embedded wire. During use, the disposable insert secures to the reusable shell with the Velcro patches, and then the combined system is attached to the infant using the Velcro tabs. When the insert is soiled following a PP event, the Velcro tabs are undone and the entire diaper is removed. The disposable insert is then removed from the reusable shell, and thrown away.

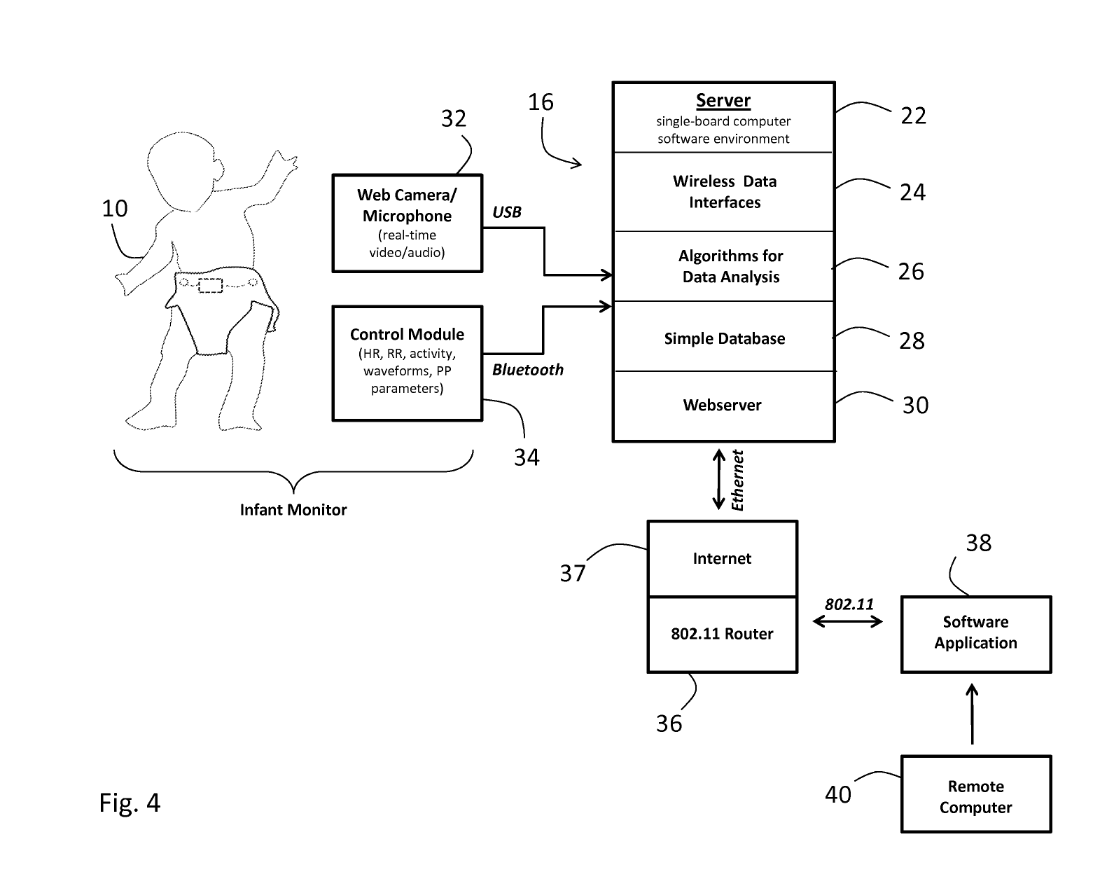

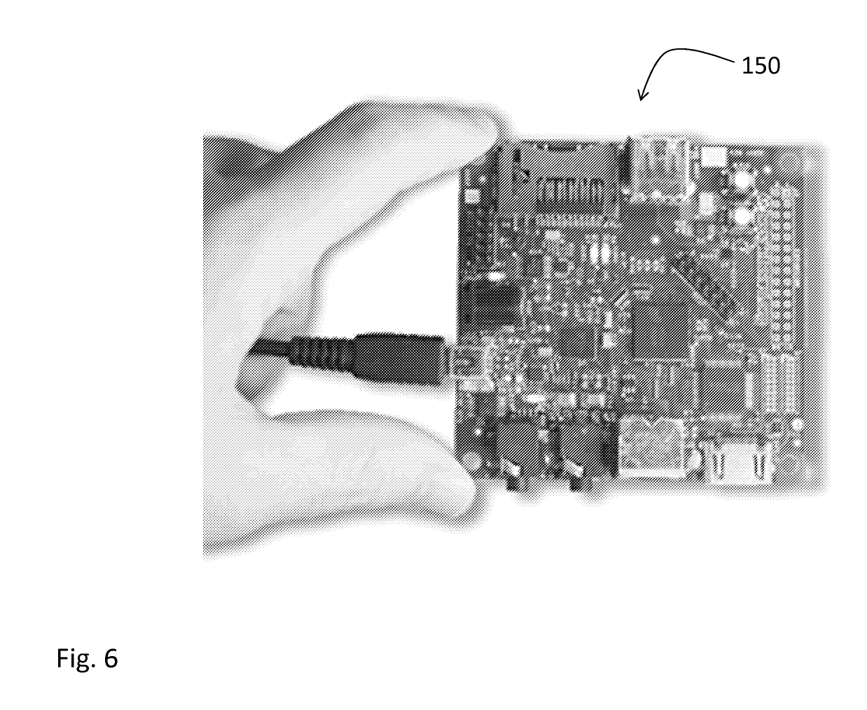

FIGS. 4-6 show how the monitoring system 16 integrates with the control module 14 within the electronic diaper 12 to monitor an infant. As shown in FIG. 6, the monitoring system 16 is based on a single-board computer 150 that serves as a central controller. Preferably the single-board computer is the Beagleboard computer (model 1234), available at www.beagle board.org, although any similar computing system can be used for this application. The single-board computer features a microprocessor and random access memory, thus allowing it to be programmed with a software system shown schematically in FIG. 4. More specifically, the software system programmed onto the monitoring system 16 includes: i) a server 22 that controls its operation; ii) a set of wireless data interfaces 24 that receive and transmit data through WiFi and Bluetooth interfaces; iii) a collection of algorithms 26 that analyze waveforms measured from the infant to determine vital signs and other properties; iv) a simple database 28 for storing information collected from the infant; and v) a webserver 30 for availing this information to a network. To collect video and audio signals from the infant, the monitoring module 16 also integrates with a web camera/microphone system 32, which is typically a conventional system that plugs directly into the single-board computer. The web camera/microphone system 32 records real-time digital images and sounds from the infant, and sends these to embedded software within the monitoring module for processing. Processing can include simple image-processing techniques, for example, that reduce the size of the image to make it easier to display on the software application. Additionally, the image may be analyzed to estimate vital signs, such as HR and RR, as well as motion-related properties, such as posture and degree of motion. Determination of RR from an image can also be done by analyzing the image to detect slight motions of the patient's chest caused by respiratory effort. Similarly, posture can be determined by analyzing the image and comparing it to pre-determined image models wherein the posture is known and well-defined.

The wireless data interfaces 24 include two separate wireless systems: 1) a Bluetooth transmitter that, during use, is paired to the Bluetooth transceiver in the control module; and 2) a transceiver operating on an 802.11-based protocol that connects the single-board computer 150 to a local-area network. Other similar transmitters and transceivers can also be used. These systems can be in the form of ‘daughter’ circuit boards that connect to the single-board computer, or USB-based peripherals that simply plug into USB ports available on this system. Typically each wireless system will have an associated software driver that is loaded onto the single-board computer to facilitate its operation.

The server 22 features a software ‘packet parser’ that deconstructs packets sent by the control module to the monitoring module. Source code for the packet parser is included, e.g. in Appendix A. The packet parser extracts numerical data from the packets so it can be processed by the algorithms for data analysis 26, as described in more detail below. During normal operation a packet is sent from the control module every 10 seconds, and is then parsed and immediately processed by the monitoring module. Ideally a latency of about 2-5 seconds separates an actual physiological event on the infant, and when this event is determined by the monitoring module.

The software system features a beat-picking algorithm that analyzes ECG waveforms to determine HR along with cardiac abnormalities, such as ventricular tachycardia (VTAC), ventricular fibrillation (VFIB), and cardiac arrhythmias. Conventional beat-picking algorithms that may be used for this application include the Pan-Tompkins algorithm, as described in the following article, the contents of which have been previously incorporated herein by reference: A Real-Time QRS Detection Algorithm, Pan et al., IEEE Transactions of Biomedical Engineering, Vol. BME-32, No. 3, March, 1985. Such an algorithm allows detection of normal and ventricular beats, and can effectively yield a value for HR that is stored in memory for later processing.

To determine RR, the software system includes a breath-picking algorithm that analyzes breathing-induced modulations of the motion waveform. Such an algorithm is defined, for example, in the following article, the contents of which are incorporated herein by reference: An Open-Source Algorithm to Detect Onset of Arterial Blood Pressure Pulses, Zong et al., Computers in Cardiology, Vol. 30, 2003. Similarly, the software system includes an algorithm for analyzing all three of the motion waveforms to determine the infant's posture and crawling behavior.

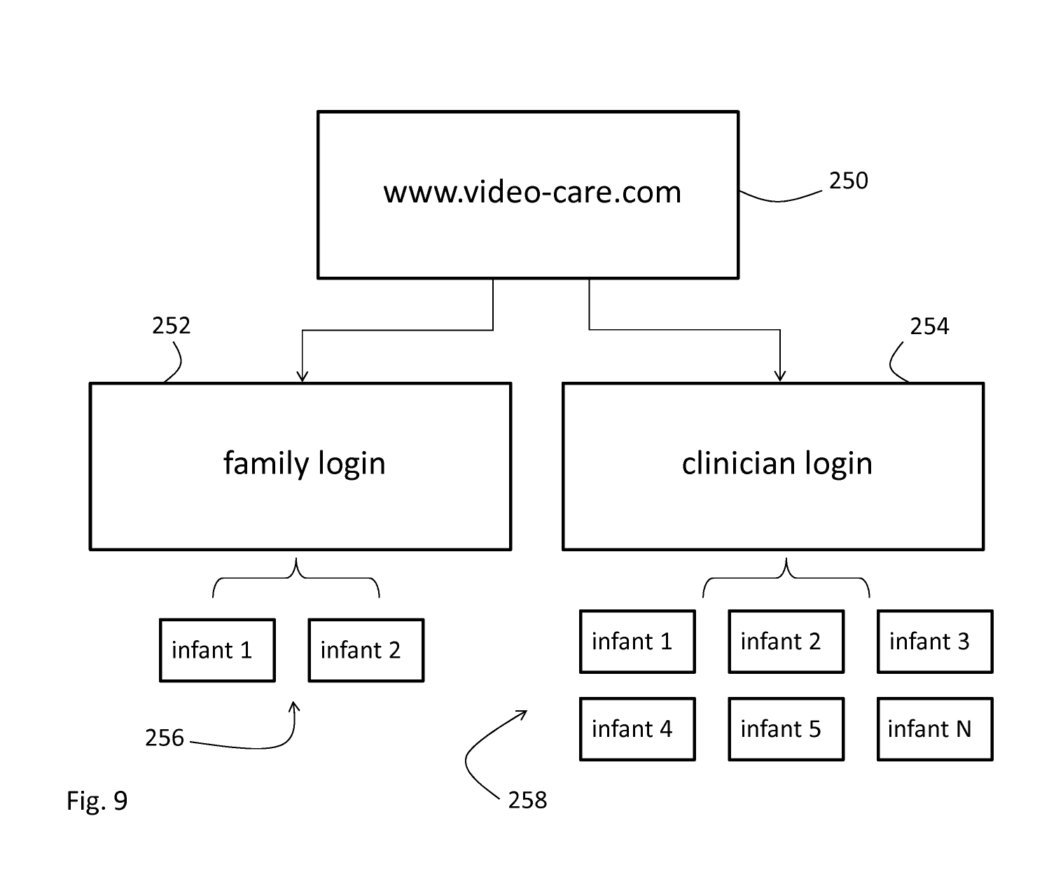

A simple database 28 includes time/date stamps for each packet received by the monitoring module, and thus allows numerical and waveform data to be read out and analyzed at a later time. For example, using a website similar to that shown in FIG. 9, these data could be analyzed by a pediatrician to perform a ‘virtual checkup’ on the infant. The database typically stores information in flash memory for a period of several months.

A webserver 30 running on the single-board computer avails data collected from the infant over the Internet 37, which can be accessed using standard methodologies using a conventional WiFi router 36. Such a router 36, for example, would be the one already present in the parents' home. With this system the parent can download the software application 38 from a website, and load it onto an existing remote computer 40 to monitor the infant. This system then displays numerical, waveform, video, and other information, as described in detail herein.

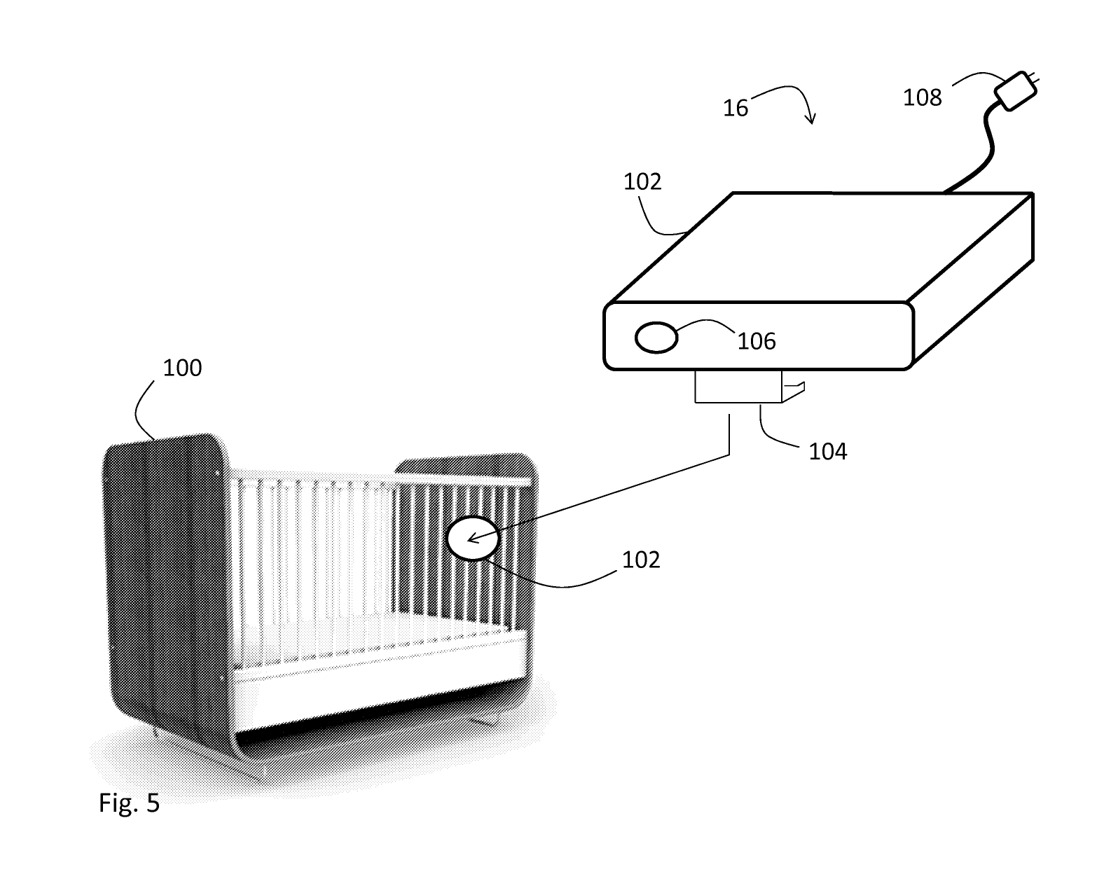

FIG. 5 shows a physical embodiment of the monitoring module 16. A plastic housing 102 typically encases the single-board computer and includes an opening 106 for the lens of the web camera, and a standard plug 108 that allows it to receive power from a wall outlet. The plastic housing 102 also features a simple spring-loaded clip 104 that attaches it, for example, to the side of a crib 100. In this way the monitoring module 16 can be used to monitor the infant in a variety of settings, e.g. at home, work, daycare, or at the house of another family member. The spring-loaded clip 104 can also attach to other structures (e.g. a table, stand) so that the infant can be monitored outside the crib 100.

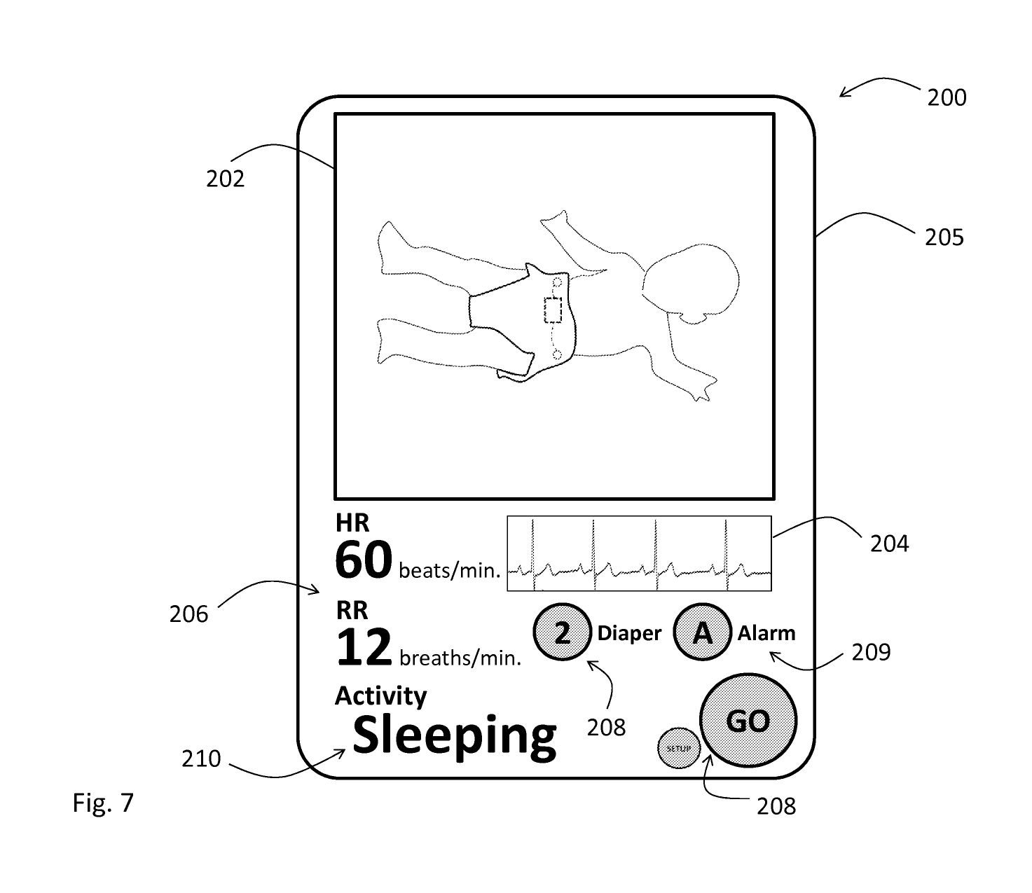

FIG. 7 shows an image of a user interface 200 rendered by the downloadable software application. An external, remote computer 205, such as a laptop computer, desktop computer, tablet computer, or cellular telephone, operates the user interface 200 to display content received from the webserver within the monitoring module. It features a real-time image 202 of the infant, which is typically updated several times each second, as captured with the web camera. Audio associated with the image 202 typically plays through the speaker system of the remote computer. The interface also includes a section 206 dedicated to vital signs. The section 206 typically includes time-dependent waveforms 204, e.g. ECG waveforms and/or motion-related waveforms associated with the infant's RR. In place of these waveforms the user interface 200 may display a graphic that is less medically oriented but more understandable to a non-clinician, e.g. a beating heart, expanding pair of lungs, or simply a time-dependent graphical component that's indicates heart or respiration rate.

The section 206 typically includes numerical values of HR, RR, and possibly other vital signs, e.g. skin temperature, SpO2, and/or blood pressure. Such sensors are well known in the art. For example, SpO2 is typically measured by analyzing PPG waveforms measured simultaneously using red and infrared LEDs using techniques known in the art. These waveforms can also be measured to determine pulse rate, which is directly related to HR. The LEDs, for example, can be located close to the infant's skin to optimize optical coupling. Skin temperature can be measured with a separate temperature sensor, similar to that used to detect a PP event, which directly contacts the infant's skin. Blood pressure can be measured with a standard pneumatic cuff or by simultaneously measuring ECG and PPG waveforms. Here, a time difference between features in these waveforms, often called pulse transit time, is inversely related to blood pressure. RR can also be measured by analyzing a slowing varying envelope of one or both of the ECG and PPG waveforms. This envelope is directly related to RR, and can be processed using, e.g., a low-pass filter applied to these waveforms.

Waveforms associated with these parameters may be displayed as well. As described above, in a preferred embodiment, numerical values of the vital signs are calculated with algorithms operating on the monitoring module. Here, in order to optimize battery life, the control module on the diaper simply collects associated waveforms and routes them to the monitoring module for further analysis.

The user interface 200 also includes a section 210 that indicates the infant's activity level and/or posture, e.g. if the infant is standing up, sleeping, or lying done in the crib. Postural states are determined, as described above, using algorithms operating on the monitoring module that process time-dependent waveforms measured by the accelerometer. Whether or not the infant is sleeping is determined by processing the postural state (lying down), a decreased HR, and an RR characterized by deep, steady breaths. Activity states such as crawling can be determined by analyzing the motion waveforms, as described above. In all cases such states can be indicated in section 210 by text, a simple icon, or a combination of both.

Importantly, the user interface 200 includes a section 208 that indicates if a PP event has occurred, i.e. if the infant has urinated or defecated. Typically, the section simply includes a number to describe these events, with, fittingly, the number ‘1’ indicating that the infant has urinated, and the number ‘2’ indicating that the infant has defecated. These numbers can also be replaced by simple icons indicating the events.

Off-the-shelf sensors do not readily exist for measuring PP events, and thus existing sensors and algorithms for processing data generated by them must be used as replacements. For example, conventional temperature sensors coupled with numerical signal-analysis algorithms may be used in this application. The temperature sensor is typically a digital sensor, such as the TMP112 sensor manufactured by Texas Instruments, which communicates with the microprocessor in the control module through a 2-wire interface and a 4-wire cable. The temperature sensor is typically mounted in the reusable shell, as shown in FIG. 2B, so that it can to be separated from the infant's skin while still being coupled to the disposable insert, thus allowing temperature changes can be easily detected.

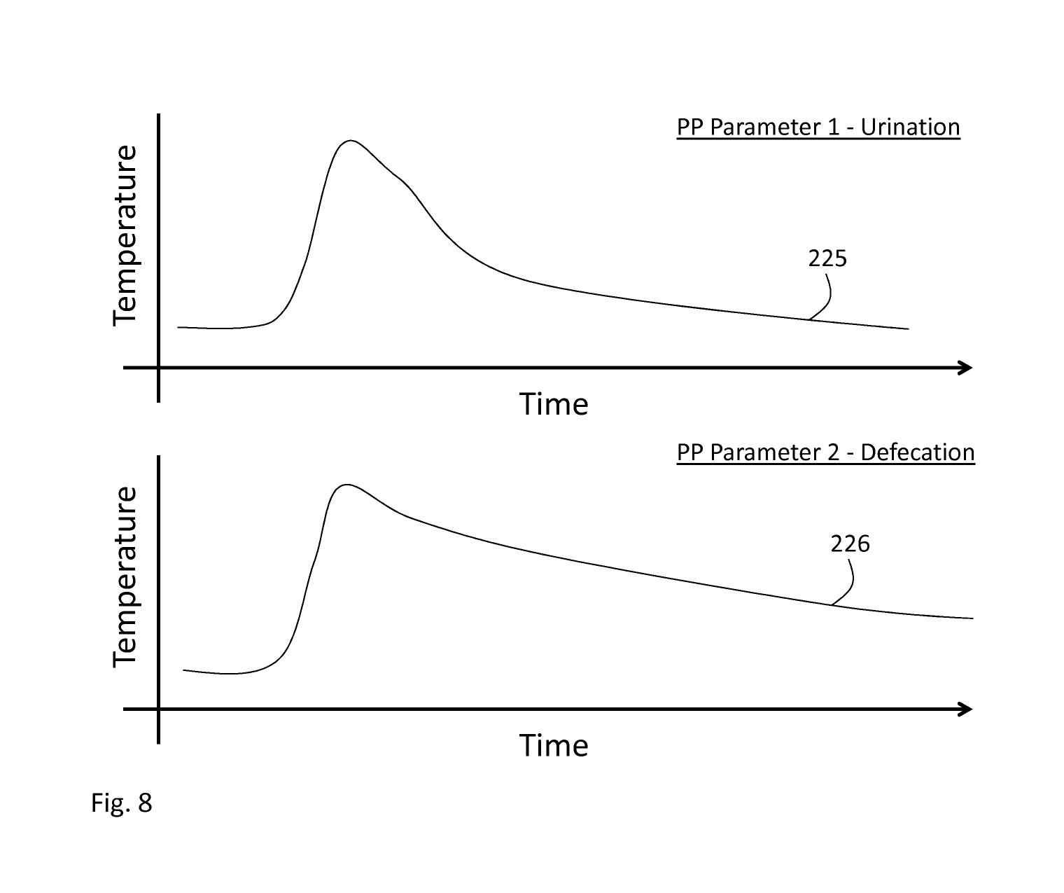

Without being bound to any theory, signal-analysis algorithms can analyze time-dependent temperature profiles generated by the temperature sensors to determine a specific PP event. FIG. 8 shows, for example, time-dependent temperature waveforms 225, 226 associated with, respectively, number ‘1’ and ‘2’ events. During operation, such waveforms are only transmitted from the control module in the electronic diaper to the monitoring module when a simple algorithm operating on the control module indicates that a significant rise in temperature has occurred in the disposable insert, thus signifying a number ‘1’ or ‘2’ event. Not transmitting temperature waveforms in the absence of such events decreases the amount of power consumed by the control module, thus increasing battery life. When such an event does occur, the control module immediately transmits the measured waveform, where it is then analyzed by the monitoring module in detail as described above.

In the case of urination (i.e. a PP parameter of ‘1’), the temperature sensor will detect a rapid rise in temperature as urine is absorbed by the disposable insert in the diaper. As shown by the waveform 225 in the top portion of the figure, the temperature quickly decreases as the insert soaks up and dissipates the urine. The time-dependent decrease in temperature associated with this event is typically associated with an exponential decay, with the time constant of the decay typically associated with the amount of urine.

A PP parameter of ‘2’, which indicates defecation, also results in a detectable change in temperature in the disposable insert. But as shown by the waveform 226 in the bottom portion of the figure, this is characterized by a relatively slow time-dependent change in temperature as compared to a number ‘1’ event. More specifically, an infant defecating into a diaper causes a rapid increase in temperature in the disposable insert, much like a number ‘1’ event. However, because the feces cannot be fully absorbed, the temperature in the disposable insert stays at a relatively high level for an extended period of time. This means the time constant associated with a number ‘2’ event is much longer than that associated with a number ‘1’ event.

Referring again to FIG. 7, the user interface 200 includes a section 209 that indicates an alarm state, along with a separate set of pages (not shown in the figure) wherein a user can enter alarm-related properties using the remote computer. In a preferred embodiment, alarms (e.g. audio or visual alarms) are controlled and instigated by hardware and software operating on the remote computer, as opposed to similar components operating on the control or monitoring modules. For example, the user interface can include sections where the user enters simple alarm ‘thresholds’ that are triggered when a parameter measured by the control module and sent to the remote computer exceeds the threshold. In one embodiment, for example, the user may enter ‘high’ and ‘low’ values associated with vital signs such as HR, RR, and temperature. Here, an alarm is generated when one or more of the infant's vital signs exceeds the thresholds (i.e. trends higher than the ‘high’ threshold, or lower than the ‘low’ threshold) for a predetermined period of time. As described above, the alarm can be an audio or visual signal generated by the remote computer.

In related embodiments, the user can enter non-threshold alarm parameters associated with the infant's posture, whether or not the infant is sleeping (as determined, for example, by a combination of posture, HR, and RR as described above), and whether or not a PP event has occurred. For example, the user may enter parameters that causes an alarm to sound if the infant stands up in their crib, if they are sleeping on their back (as opposed to their stomach), or if they have soiled their diaper.

The user interface may also include a feature (e.g. a simple software button) that allows a user to ‘activate’ a pre-determined alarm, e.g. an alarm associated with a serious medical condition that may occur in the infant. For example, ‘sudden infant death syndrome’ (SIDS) is not fully understood in clinical medicine, but is assumed to occur when an event related to apnea, i.e. a sudden cessation of breathing, occurs in the infant. SIDS may thus occur when RR rapidly drops to a low or non-measurable value, and HR trends to a high level. Trends in both RR (indicating a systematic decrease in this value) and HR (indicating a systematic increase) can be thus analyzed to estimate the onset of SIDS, and thus trigger a predetermined alarm. In general, the system described herein can be used to analyze trends in both HR and, more importantly RR, to help predict the onset of a possibly life-threatening condition before it actually occurs. During such a situation, an alarm operating on the remote computer can sound, thus alerting the infant's parents and causing them to react accordingly.

In another embodiment, as shown in FIG. 9, the system includes a web-based interface 250 (in this case www.video-care.com) that features a ‘family’ interface 252 and a ‘clinician’ interface 254. Access to a particular interface is determined by a user name and password, which is entered into the web-based interface using standard means. The family interface 252 is typically associated with infants 256 belonging to a particular family, and would render much of the same content that is shown in FIG. 7. This would allow, for example, remote family members to view real-time images of the infant, or check on the infant's vital signs, posture, activity level, or trends in these parameters. In contrast, the clinician interface is typically associated with a group of infants 258, and would be viewed by a clinician (e.g. a pediatrician) to perform a ‘virtual check-up’ on the infant. For example, the clinician could view ECG waveforms and abnormalities in heartbeats associated with these waveforms (e.g. premature ventricular beats), trends in HR and RR, sleeping behavior, and other features that indicate the status of the infant's physiology.

Other embodiments are within the scope of the invention. In general, a specific intent of the invention is to combine some of the functionality of medical-grade vital sign monitors with that of consumer computing platforms, and bring this solution into the home to monitor infants. Thus the invention can include many of the capabilities of monitors which are normally used in the hospital or with high-end telemedicine systems. For example, the electronic diaper can measure high-quality ECG waveforms, which can then be sent through the Internet to a web-based system that can be viewed by a pediatrician and used to monitor the infant's cardiac performance. Or trends in the infant's vital signs can be transported and analyzed in a similar manner to diagnose certain medical conditions. Motion-related properties, such as how often an infant is crawling, or their posture, can also be analyzed in this way to determine if the infant's motor skills are developing in a normal way. In general, the invention described herein allows an infant to be monitored in the comfort of home in much the same way that it could be monitored in the hospital.

The infant-monitoring system of the invention can feature a high-end computing platform that connects to the Internet, and thus all the features of such systems can be incorporated into the invention to help improve infant monitoring. For example, using an accompanying web-based system, the electronic diaper and monitoring module can be deployed to monitor an infant in one location (e.g. a daycare center), while the remote computer can be deployed in virtually any other location with Internet connectivity so that the infant can be observed. This allows, for example, the infant to be viewed by family members, medical professionals, and research scientists. In another embodiment, the remote computer can be used to download sounds, music, or educational content from the Internet, and then transfer these to the monitoring module for playback.

In other embodiments, the electronic diaper can be deployed in a form factor other than that described above. For example, rather than featuring a relatively large reusable shell and a relatively small disposable insert, the electronic diaper can consist of a disposable diaper similar to those available today (e.g. diapers made under the Huggies or Pampers brand) that includes a small, discrete insert for the monitoring module. Here, the disposable diaper may include integrated electrodes (composed e.g. of materials such as conductive rubber or conductive fabric) that connect to the control module through a simple connector. In this embodiment, the control module is typically encased in a durable, waterproof housing that allows it to withstand day-to-day abuse by the infant. In still other embodiments, the control module is integrated with a reusable cloth diaper that is typically washed in between uses. In general, the scope of the invention extends to any form factor that combines a diaper with a control module described herein, and then couples the control module to a monitoring module and downloadable software interface as described herein.

Other embodiments of the invention are within the scope of the following claims.

What is claimed is:

1. A system for monitoring a patient, comprising:
a garment configured to attach to the patient;
a control module connected to the garment comprising: i) a first sensor configured to measure at least one of heart rate or a parameter used to determine heart rate; ii) a second sensor configured to measure at least one of respiration rate or a parameter used to determine respiration rate; iii) a third sensor configured to monitor a parameter indicating if the patient urinates and/or defecates and iv) a wireless transmitter configured to receive and wirelessly transmit information from the first, second, and third sensors;
a monitoring module configured to receive information from the first, second, and third sensors through the wireless transmitter, the monitoring module comprising; i) a processing component configured to process information generated by at least one of the first, second, and third sensors; and ii) a computing component configured to make content determined by the processing component available on a network; and
a software application operated on a remote computer and configured to connect to the network and receive and them display the content availed by the computing component, or parameters calculated therefrom.

2. The system of claim 1, wherein the garment is a diaper, wherein the diaper optionally comprises an outer component configured to attach to the lower portion of the patient's torso, and an inner component comprising an absorbent material configured to contact the patient's skin.

3. The system of claim 2, wherein the diaper further comprises at least one conductive electrode attached to the outer component and configured to contact the patient's skin, wherein the first sensor optionally comprises an ECG sensor that connects to the at least one conductive electrode to receive an electrical signal.

4. The system of claim 3, wherein the diaper further comprises two conductive electrodes, each attached to the outer component and positioned to contact separate portions of the patient, each of the conductive electrodes connected to the ECG sensor and configured to provide a unique electrical signal that the ECG sensor collectively processes to determine an ECG waveform.

5. The system of claim 1, wherein the first sensor comprises an optical sensor, wherein the optical sensor optionally comprises a photodiode and a light source, and wherein the light source is optionally a light-emitting diode.

6. The system of claim 5, wherein the optical sensor is configured to measure a photoplethysmogram from the patient.

7. The system of claim 6, further comprising an algorithm configured to analyze the photoplethysmogram to determine a heart rate corresponding to the patient.

8. The system of claim 1, wherein the second sensor is an accelerometer, wherein the accelerometer is optionally configured to measure a time-dependent waveform indicating the patient's motion.

9. The system of claim 8, wherein the time-dependent waveform indicates respiratory-induced motion of the patient's torso.

10. The system of claim 9, wherein the time-dependent waveform indicates motion measured along an axis of the accelerometer that is approximately normal to the patient's belly.

11. The system of claim 10, wherein the axis is within +/−30 degs. of a normal vector extending from the patient's belly.

12. The system of claim 1, wherein the second sensor comprises an electrode, wherein the electrode optionally measures an impedance from the patient that varies with respiration rate.

13. The system of claim 12, wherein the second sensor comprises an impedance pneumography sensor.

14. The system of claim 1, wherein the third sensor comprises a thermal sensor, wherein the thermal sensor is optionally configured to measure a digital temperature signal indicative of urine and/or feces from the patient.

15. The system of claim 1, wherein the third sensor comprises a moisture sensor.

16. The system of claim 1, wherein the wireless transmitter operates on a protocol based on 802.11 or 802.15.4.

17. The system of claim 16, wherein the wireless transmitter comprises a Bluetooth low-energy transmitter.

18. The system of claim 1, wherein the processing component comprised by the monitoring module comprises a computer.

19. The system of claim 18, wherein the processing component comprised by the monitoring module comprises a computer operating an algorithm.

20. The system of claim 18, wherein the computer is a single-board computer.

21. The system of claim 19, where the algorithm is a beat-picking algorithm configured to analyze ECG waveforms from the first sensor to measure heart rate.

22. The system of claim 21, wherein the beat-picking algorithm is a Pan-Tompkins algorithm or a derivative thereof.

23. The system of claim 19, wherein the algorithm is a breath-picking algorithm configured to analyze waveforms modulated by respiration rate from the second sensor to determine a respiratory rate.

24. The system of claim 23, wherein the breath-picking algorithm is a slope-summing algorithm or a derivative thereof.

25. The system of claim 19, wherein the algorithm is configured to process information from a thermal sensor to determine if the patient has urinated or deficated.

26. The system of claim 25, wherein the algorithm is a curve-fitting algorithm.

27. The system of claim 25, wherein the algorithm comprises taking a mathematical derivative of a time-dependent waveform generated by the thermal sensor.

28. The system of claim 1, wherein the monitoring module further comprises a camera.

29. The system of claim 28, wherein the monitoring module further comprises a web camera that captures real-time video images of the patient.

30. The system of claim 1, wherein the computing component is a computer operating a software program.

31. The system of claim 30, wherein the computer is a single-board computer.

32. The system of claim 30, wherein the software program is configured to operate a webserver.

33. The system of claim 1, wherein the content determined by the processing component is at least one of an image, a vital sign, a time-dependent physiological waveform, a motion waveform, a motion-related parameter, a posture, an indication if the patient is sleeping, and an indication if the patient has urinated or deficated.

34. The system of claim 1, wherein the network comprises a wireless network.

35. The system of claim 1, wherein the network comprises the Internet.

36. The system of claim 1, wherein the software application is configured to operate on a remote computer, wherein the remote computer is optionally selected from the group consisting of a desktop computer, laptop computer, tablet computer, cellular telephone, or smartphone.

37. The system of claim 36, wherein the software application is configured to be downloaded from a website operating on the Internet.

38. The system of claim 36, wherein the software application comprises a graphical user interface that displays an image and at least one of a vital sign, a time-dependent physiological waveform, a motion waveform, a motion-related parameter, a posture, an indication if the patient is sleeping, and an indication if the patient has urinated or deficated.

39. The system of claim 36, wherein the software application includes a section to set and/or select alarm parameters.

40. The system of claim 39, wherein the section includes an interface that allows a user to enter alarm thresholds associated with vital sign values.

41. The system of claim 39, wherein the section includes an interface that allows a user to enter alarm parameters associated with whether or not the patient has urinated or deficated.

42. The system of claim 39, wherein the section includes an interface that allows a user to enter alarm parameters associated with whether or not the patient is sleeping.

43. The system of claim 39, wherein the section includes an interface that allows a user to enter alarm parameters associated with the patient's posture.

44. The system of claim 39, wherein the section includes an interface that allows a user to enter alarm parameters associated with the patient's motion.

45. The system of claim 1, further comprising an Internet-based system that integrates with the software application.

46. The system of claim 45, wherein the Internet-based system is a website.

47. The system of claim 46, wherein the website comprises a first user interface associated with a family member associated with the patient, and a second user interface associated with a medical clinician.

48. The system of claim 47, wherein the second user interface is associated with a plurality of patients.
